(19) United States
(12) Patent Application Publication
(10) Pub. No.: US 2022/0370393 A1
(43) Pub. Date: Nov. 24, 2022

(54) METHOD AND COMPOSITION FOR INCREASING MUSCLE PROTEIN SYNTHESIS

(71) Applicant: Capsugel Belgium NV, Bornem (BE)

(72) Inventors: Aouatef Bellamine, Morristown, NJ (US); Stanislaw Glab, Greenwood, SC (US); Shane Durkee, Morristown, NJ (US)

(21) Appl. No.: 17/765,173

(22) PCT Filed: Oct. 16, 2020
(86) PCT No.: PCT/US2020/056033

§ 371 Date: Mar. 30, 2022

Related U.S. Application Data
(60) Provisional application No. 63/021,766, filed on May 8, 2020.
(60) Provisional application No. 62/915,709, filed on Oct. 16, 2019.

(51) Int. Cl. A61K 31/205; A61K 9/48; A61P 21/06

(52) U.S. Cl. CPC A61K 31/205; A61K 9/4816; A61P 21/06

(57) ABSTRACT

A method and composition for increasing muscle protein synthesis in mammals is disclosed. In one embodiment, the mammals are administered a protein building composition comprising at an amino acid derivative including L-carnitine and a nitrogenous organic acid including creatine. In a particular embodiment, the creatine is in an acid resistant capsule delivery form. The ratio of L-carnitine to creatine can be from about 10:1 to about 1:1. The protein building composition can be administered in a monolithic enteric capsule.

### RELATED APPLICATIONS

The present application is based on and claims priority to U.S. Provisional Patent application Ser. No. 62/915,709, filed on Oct. 16, 2019, and U.S. Provisional Patent application Ser. No. 63/021,766, filed on May 8, 2020, both of which are incorporated herein by reference.

### BACKGROUND

Protein synthesis is important for mammals of all ages, including both children and adults. Muscle protein synthesis is activated by the mammalian target of rapamycin (mTOR) pathway. The mTOR pathway senses and responds to changes in amino acids and growth factors such as insulin-like growth factor (IGF-1). In particular, the mTOR complex phosphorylates ribosomal protein S6K, which in turn phosphorylates ribosomal protein S6 and substrates eIF4B and 4EBP1, consequently promoting mRNA translation and protein synthesis. The proteins involved in the mTOR pathway can serve as biomarkers for protein synthesis.

Accordingly, there exists a need for a supplement containing ingredients or components that can increase muscle protein synthesis, which can lead to improved functional strength and/or overall quality of life in mammals without having any substantial adverse effects on other body functions. A need also exists for a simpler and less expensive supplement that can improve muscle protein synthesis.

Furthermore, certain compounds that may be administered to improve muscle health, such as creatine, may be degraded by low pH environments, such as those in the stomach. In order to address this degradation, higher concentrations of creatine have been administered or included in certain supplements in order to account for any in vivo degradation. Furthermore, given this degradation, administration of supplements containing creatine may not provide all the desired benefits. Accordingly, there exists a need for a supplement and/or dosage formulation that contains an effective amount of creatine that does not require higher amounts of creatine to compensate for gastrointestinal degradation.

### SUMMARY

The present disclosure is generally directed to a method and composition for increasing protein synthesis in mammals. In accordance with the present disclosure, the method comprises the step of administering to a mammal an effective amount of a protein building composition. In one embodiment, the protein building composition may comprise an amino acid derivative, such as L-carnitine and a nitrogenous organic acid, such as creatine. Furthermore, the protein building composition may contain creatine in lipid microparticulate form. Of particular advantage, the protein building composition may contain a lower amount of creatine than other supplements due to the fact that the lipid multiparticulate form of creatine disclosed herein undergoes less degradation in the gastrointestinal tract when administered. Also, it was discovered that the combination of L-carnitine and creatine according to embodiments disclosed herein provided synergistic results with respect to an increase muscle protein synthesis. Accordingly, lower amounts of creatine can be administered given the synergies provided herein.

In certain embodiments, the creatine comprises a lipid multiparticulate (LMP) creatine also known as LMP-creatine. In some embodiments, one or more of the components of the protein building composition, such as creatine, may be in lipid multiparticulate form. For example, in certain embodiments the creatine may be dispersed in a lipid matrix. In one embodiment, the lipid matrix compositions are liquid at ambient temperature. In one embodiment, the lipid matrix compositions are semi-solid at ambient temperature. In one embodiment, creatine is molecularly dispersed in the lipid matrix. In some embodiments, the creatine in lipid multiparticulate form comprises one or more particles containing a lipid matrix having creatine dispersed therein. In one embodiment, the creatine in lipid multiparticulate form may include one or more particles having a mean diameter ranging from about 40 μm to about 3000 μm. In some embodiments, the lipid matrix comprises from about 10% to about 60% by weight of creatine. In some embodiments, the lipid matrix comprises from about 15% to about 25% by weight of stearyl alcohol. In some embodiments, the lipid matrix may contain from about 10% to about 20% by weight of stearic acid. In some embodiments, the lipid matrix may contain from about 10% to about 20% by weight of a wax. In some embodiments, the lipid matrix comprises from about 1% to about 3% by weight of a lecithin.

In one embodiment, the amino acid derivative comprises L-carnitine. In a particular embodiment, the amino acid derivative comprises L-carnitine L-tartrate. L-carnitine may be present in a substantially pure crystalline form or as salts, metabolites, and/or lipid and non-lipid derivatives of L-carnitine. In some embodiments, the L-carnitine is in lipid multiparticulate form. In one embodiment, L-carnitine is molecularly dispersed in a lipid matrix. In some embodiments, the L-carnitine in lipid multiparticulate form comprises one or more particles containing a lipid matrix having L-carnitine dispersed therein. In one embodiment, the L-carnitine in lipid multiparticulate form may include one or more particles having a mean diameter ranging from about 40 μm to about 3000 μm.

In some embodiments, the ratio of L-carnitine to creatine is from about 10:1 to about 1:3. In some embodiments, the ratio of L-carnitine to creatine is from about 5:1 to about 1:2. In some embodiments, the ratio of L-carnitine to creatine is from about 3:1 to about 1:1. In some embodiments, the ratio of L-carnitine to creatine is from about 2:1 to about 1:1.

In some embodiments, the protein building composition is contained in a monolithic enteric capsule. The enteric capsule may be coated or formulate such that less than about 10% of the protein building composition is released form the monolithic enteric capsule after about 2 hours in a pH of about 1.2. In certain embodiments, the enteric capsule may be coated or formulated such that less than about 80% of the protein building composition is released from the monolithic enteric capsule after about 30 min at a pH of about 6.8. In certain embodiments, the enteric capsule may be coated or formulated such that more than about 95% of the protein building composition is released in the intestine.

The present disclosure is also directed to a composition for increasing muscle protein synthesis containing a protein building composition that contains an amino acid derivative comprising L-carnitine and a nitrogenous organic acid comprising creatine, wherein the creatine is in lipid multiparticulate form, and further wherein the ratio of L-carnitine to creatine is from about 5:1 to about 1:1.

The present disclosure is also directed to a nutraceutical composition containing a monolithic enteric hard capsule filled with a protein building composition that contains an amino acid derivative comprising L-carnitine and a nitrogenous organic acid comprising creatine, wherein the creatine is in lipid multiparticulate form. In some embodiments, the ratio of L-carnitine to creatine is from about 5:1 to about 1:1.

The present disclosure is also directed to a method of producing a nutraceutical composition containing the protein building composition. The method can include the steps of providing a protein building composition and filling a monolithic enteric hard capsule with the protein building composition.

In one aspect, the present disclosure is directed to a composition for increasing muscle protein synthesis. The composition comprises a protein building composition comprising an amino acid component and a nitrogenous organic acid component. The amino acid component comprises L-carnitine or a derivative thereof. The nitrogenous organic acid component comprises creatine or a derivative thereof. In accordance with the present disclosure, the protein building composition is contained in an acid resistant capsule shell. The capsule shell exhibits resistance to leakage for at least one hour at a pH of 1.2 in a USP-30 simulated gastric fluid. The capsule shell, for instance, can be made from a material comprising hydroxypropyl methylcellulose. For example, the capsule shell can be made from a material comprising hydroxypropyl methylcellulose in combination with a gum, such as gellan gum. In one aspect, the weight ratio between the gum and the hydroxypropyl methylcellulose is from about 4.5 to about 15 parts of the gum per 100 parts of the hydroxypropyl methylcellulose.

In one embodiment, the amino acid component and the nitrogenous organic acid component can both be solids that are blended together and contained within the capsule shell. In fact, in one embodiment, the protein building composition can comprise particles in which each particle contains both the amino acid component and the nitrogenous organic acid component. Alternatively, the amino acid component can be separate from the nitrogenous organic acid component.

Capsules used in accordance with the present disclosure generally include two cooperating parts. For instance, the capsule shell can have an elongated body formed from a first part or component that cooperates with and overlaps with a second part or component. In one aspect, the capsule can be banded. A banded capsule is one that has an additional closure or seal where the two parts of the capsule come together. The use of a banded capsule can further prevent acid breakdown of the capsule in the stomach. Banded capsules also provide a tamper evident seal and prevents the capsules from leaking, especially when containing any liquid components.

Alternatively, the protein building composition can comprise a suspension. For example, the L-carnitine or derivative thereof can comprise a liquid into which the creatine is suspended. The creatine suspended within the protein building composition, for instance, can be in liquid multiparticulate form or pure form and can comprise solid particles.

The liquid containing the L-carnitine or derivative thereof can, in one aspect, comprise the L-carnitine or derivative thereof dissolved in a liquid carrier, such as a polar solvent. The liquid carrier, for instance, can comprise water. In addition to the liquid carrier, the liquid can also contain various other components, such as a pH adjusting agent. The pH adjusting agent, for instance, can be added in order to maintain the pH of the liquid above 4, such as above 5.

The present disclosure is also directed to a method for increasing muscle protein synthesis. The method comprises administering to a mammal an effective amount of a protein building composition. The protein building composition comprises an amino acid component combined with a nitrogenous organic acid component. The amino acid component comprises L-carnitine or a derivative thereof. The nitrogenous organic acid component comprises creatine or a derivative thereof. In accordance with the present disclosure, the protein building composition is contained in an acid resistant capsule shell as described above. The capsule shell can exhibit resistance to leakage for at least one hour at a pH of 1.2 in a USP-30 simulated gastric fluid.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

The Figures are graphical representations of the results obtained in the example described below.

### Definitions

The term “MET,” or “metabolic equivalent,” means the ratio of the rate of energy expended during an activity to the rate of energy expended at rest. A body at rest has a rate of energy expenditure of 1 MET. If a body performs a 2 MET activity, the body has expended 2 times the energy used by the body at rest. The term “physical activity” means bodily movement with an energy expenditure rate equal to or greater than 3 MET. Non-limiting examples of “physical activity” include bicycling, sexual activity, giving birth, jogging, walking at a speed of about 3 mph or greater, calisthenics, jumping rope, running, sprinting, or any combinations thereof.

In one embodiment, “physical activity” can mean a negative energy balance in the mammal, such as weight loss, diets, aging, gestation, and lactation.

The term “physically active” means regularly participating in body movements with an energy expenditure rate of greater than or equal to 3 MET.

In one embodiment, “physically active” can mean regularly meeting medically recommended standards for amount, intensity, and type of physical activity performed by a mammal.

The terms “sedentary” or “sedentary activity” mean participating mainly or exclusively in body movements with an energy expenditure rate of less than 3 MET. Non-limiting examples of “sedentary” activities include sleeping, resting, sitting or reclining, watching television, writing, working at a desk, using a computer, typing, walking at a speed of less than about 3 mph, or any combinations thereof.

In one embodiment, “sedentary” can mean a failure to regularly meet medically recommended standards for amount, intensity, and type of physical activity performed by a mammal.

The term “L-carnitine” may contain L-carnitine and derivatives and/or salts thereof. L-carnitine can include L-carnitine base or derivatives and/or salts thereof including substantially pure crystalline L-carnitine, any fatty acid derivatives thereof, acetyl L-carnitine, valeryl L-carnitine, isovaleryl L-carnitine, benzyl L-carnitine, L-leucyl L-carnitine, L-valyl L-carnitine, other L-amino acyl carnitines, salts of L-amino acyl L-carnitine, L-carnitine HCL, L-carnitine L-tartrate, L-carnitine fumarate, propionyl L-carnitine, L-carnitine phosphate, acetyl L-carnitine L-aspartate, acetyl L-carnitine citrate, acetyl L-carnitine maleate, acetyl L-carnitine phosphate, acetyl L-carnitine fumarate, propionyl L-carnitine orotate, acetyl L-carnitine orotate, butyryl L-carnitine orotate, propionyl L-carnitine fumarate, L-carnitine oxalate, L-carnitine sulfate, GPLC glycine propionyl L-carnitine, and the like.

The term “mammal” includes any mammal that may experience muscle protein synthesis and includes human, canine, equine, feline, bovine, ovine, or porcine mammals.

The phrase “effective amount” means an amount of a compound that promotes, improves, stimulates, or encourages a response to the particular condition or disorder or the particular symptom of the condition or disorder.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “functional strength” means an individual's ability to competently and safely perform daily life activities. In one embodiment, functional strength may be associated with energy, muscle potency, agility, flexibility, balance, and injury resistance.

As used herein, the term “flow point” is the temperature at which any portion of the mixture becomes sufficiently fluid that the mixture, as a whole, may be atomized. Generally, a mixture is sufficiently fluid for atomization when the viscosity of the molten mixture is less than 20,000 cp, or less than 15,000 cp, or less than 10,000 cp, less than 5000 cp, or even less than 1000 cp. The viscosity can be measured by controlled stress rheometer, which measures viscosity as a function of temperature, and may use either a shear-type or rotational rheometer. As used herein, melting point refers to the temperature that marks the midpoint of the transition from a solid crystalline or semi-crystalline state to a liquid state. As measured by DSC, the melting point is the temperature where upon heating the solid material, the maximum exothermic heat flow occurs. In general, melting point will be used in reference to relative pure single component materials such as some actives or essentially single component excipients (e.g. stearyl alcohol) and flow point will be used in reference to multi-component materials or mixtures.

The term “ambient temperature” refers to a temperature of 20° C.

As used herein, the term “semi-solid” is a solid at ambient temperature but becomes a liquid at temperatures above 30° C. or 40° C., or at body temperature.

Unless otherwise indicated, “capsule” means a container suitable for enclosing solids or liquids and includes empty capsule shells and components thereof such as caps and bodies that may be assembled together to form the capsule.

### DETAILED DESCRIPTION

It is to be understood by one of ordinary skill in the art that the present discussion is a description of exemplary embodiments only, and is not intended as limiting the broader aspects of the present disclosure.

The present disclosure is directed to a method and composition for increasing muscle protein synthesis in mammals. As will be explained below, the present disclosure is generally directed to a protein building composition that when administered to a mammal in an effective amount preserves or increases muscle mass and function by increasing muscle protein synthesis. The present disclosure is further generally directed to administering to a mammal an effective amount of a protein building composition that increases muscle protein synthesis and/or functional strength.

In one embodiment, the protein building composition comprises an amino acid derivative, such as L-carnitine, combined with at least one other component, such as creatine. The protein building composition may optionally contain other components, such as amino acids, other amino acid derivatives, organic acids, and/or nitrogenous organic acids. The carnitine included in the protein building composition may include L-carnitine.

The inventors of the present disclosure unexpectedly discovered that the components of the protein building composition disclosed herein synergistically work together to increase muscle protein synthesis and/or functional strength. Surprisingly, it has been discovered herein that the combination of L-carnitine with creatine may provide synergistic benefits not previously known or recognized. For example, it was discovered that the combination of L-carnitine and creatine may synergistically increase protein synthesis. Accordingly, administering a protein building composition containing L-carnitine combined with creatine to a mammal can increase muscle protein synthesis and/or functional strength. Of particular advantage, the above results can be achieved without administering higher dosages of creatine as required in previous compositions. In addition, the protein building composition of the present disclosure may reduce side effects as compared to other compositions.

In some embodiments, the amino acid derivative may be any suitable carnitine, such as L-carnitine and any derivatives and/or salts thereof. L-carnitine is a quaternary amine that can be biosynthesized from lysine and methionine. L-carnitine is known to promote beta-oxidation of long-chain fatty acids by facilitating their transfer across the mitochondrial membrane. L-carnitine may be present in a substantially pure crystalline form or as salts, metabolites, and/or lipid and non-lipid derivatives of L-carnitine. In addition, L-carnitine can be in the form of a solid, semi-solid, or liquid, such as a solution. When in solution, the L-carnitine can be L-carnitine or a derivative, such as acetyl L-carnitine or propionyl-L-carnitine.

In one embodiment, L-carnitine may be present in the composition at a concentration of from about 250 grams to about 700 grams. In some embodiments, the composition may contain from about 300 to about 600 grams of L-carnitine. In some embodiments, the composition may contain from about 350 to about 550 grams of L-carnitine. In some embodiments, the composition may contain from about 400 to about 500 grams of L-carnitine. In some embodiments, the composition may contain from about 500 grams of L-carnitine. For example, in some embodiments the composition may contain at least about 250 grams of L-carnitine, such as at least 300 grams, such as at least 350 grams, such as at least 400 grams, such as at least 450 grams, such as at least 500 grams, such as at least 550 grams, such as at least 600 grams, such as at least 650 grams, such as at least 700 grams. In certain embodiments, the composition may contain less than 700 grams of L-carnitine, such as less than 650 grams, such as less than 600 grams, such as less than 550 grams, such as less than 500 grams, such as less than 450 grams, such as less than 400 grams, such as less than 350 grams, such as less than 300 grams.

In certain embodiments, the L-carnitine included in the protein building composition may be formulated as lipid multiparticulates or in lipid multiparticulate form. For example, lipid multiparticulates (LMPs) are known. See for example, EP1030687, U.S. Pat. No. 6,572,892, EP1827382, U.S. Pat. Nos. 7,235,260, 7,887,844, EP1691787, U.S. Pat. No. 7,625,507. Providing L-carnitine in lipid multiparticulate form (LMP) as provided herein, may offer protection of the L-carnitine in acid rich environments, such as the stomach. Accordingly, the L-carnitine in LMP form provided herein, may offer additional benefits and provide better efficacy as compared to other compositions.

Furthermore, U.S. patent publication no. 2018/0125863, incorporated by reference herein in its entirety, describes certain oral formulations containing an active ingredient incorporated with a lipid matrix. Accordingly, in certain embodiments, the protein building composition or components of the protein building composition, such as L-carnitine, can be formulated in combination with a lipid matrix according to those disclosed in U.S. patent publication no. 2018/0125863.

Additionally, U.S. patent publication no. 2017/0354599, incorporated by reference herein in its entirety, describes certain formulations containing an active ingredient incorporated with a lipid matrix. Accordingly, in certain embodiments, the protein building composition or components of the protein building composition, such as L-carnitine, can be formulated in combination with a lipid matrix according to those disclosed in U.S. patent publication no. 2017/0354599.

In some embodiments, the L-carnitine is contained within a lipid matrix containing stearyl alcohol, stearic acid, candelilla wax, and lecithin. In certain embodiments the lipid matrix may contain from about 40% about 60% by weight of L-carnitine, such as about 50% by weight of L-carnitine. In certain embodiments, the lipid matrix may contain from about 15% to about 25% by weight of stearyl alcohol, such as about 17% by weight of stearyl alcohol. In some embodiments, the lipid matrix may contain from about 10% to about 20% by weight of stearic acid, such as from about 15% by weight of stearic acid. In some embodiments, the lipid matrix may contain from about 10% to about 20% of a suitable wax, such as candelilla wax, such as from about 15% by weight of a suitable wax. In some embodiments, the lipid matrix may contain from about 1% to about 3% of a lecithin, such as about 2% of a lecithin, such as soy lecithin.

In one embodiment, the lipid matrix composition containing L-carnitine may comprise a plurality of particles that are solid or semi-solid at ambient temperature, have a generally spherical shape, and have a mean diameter ranging from 40 μm to 3000 μm.

As described above, the protein building composition may comprise creatine. Creatine is a nitrogenous organic acid that can be biosynthesized from glycine and arginine. Creatine increases the formation of ATP and in a phosphorylated form serves as an energy reserve in skeletal muscles. Creatine can improve the physiological response to high-intensity and resistance exercise.

In one aspect, the L-carnitine or derivative thereof can be in the form of a liquid. For instance, in one aspect, the lipid matrix composition can be a liquid. Alternatively, the L-carnitine or derivative thereof can be dissolved in a liquid carrier. L-carnitine and derivatives thereof, for instance, are generally soluble in polar solvents, such as water, glycerol (primary), sorbitol, various polyols, polyethylene glycols, propylene lycol and other similar compounds that are safe to consume which are safe to consume and are capable of dissolving L-carnitine. L-carnitine, for instance, is zwitterionic.

When in a liquid form, the L-carnitine or derivative thereof can be combined with various other additives and components. For example, in addition to a liquid carrier, the liquid can contain a pH adjusting agent. L-carnitine, for instance, can display an acidic pH when dissolved in various solvents, such as water. A pH adjusting agent can be added as a buffer and/or to increase the pH of the solution. The pH adjusting agent, for instance, in one aspect, can be added to the liquid such that the resulting pH is greater than about 4, such as greater than about 4.5, such as greater than about 5 and generally less than about 8.5, such as less than about 8. Exemplary pH adjusting agents include food grade ingredients, for example, malic acid, citric acid, ascorbic acid, sodium hydroxide, sodium citrate, and the like.

Other ingredients that can be added to the liquid include pharmaceutical excipients, sweetening agents, mucilages, preservatives, and/or flavor additives

In one embodiment, the composition may include creatine and derivatives and analogs and/or salts thereof. In one embodiment, the composition may include creatine phosphate; creatine monohydrate; creatine ethyl ester; magnesium creatine chelate; creatine HCL; creatine-MG-complex (acetate); phosphocreatine-Mg-complex (acetate); creatine sugar amides and salts thereof as described in U.S. Pat. No. 8,546,369, incorporated by reference herein; (Boc)2-creatine and derivatives thereof as described in PCT Publication WO 2014/097335, incorporated by reference herein; other derivatives and salts of creatine; and any combinations thereof.

In one embodiment, creatine may be present in the composition at a concentration of from about 50 grams to about 1000 grams. In some embodiments, the composition may contain from about 100 to about 700 grams of creatine. In some embodiments, the composition may contain from about 100 grams of creatine. In some embodiments, the composition may contain from about 200 grams of creatine. In some embodiments, the composition may contain from about 500 grams of creatine. For example, in some embodiments the composition may contain at least about 100 grams of creatine, such as at least 150 grams, such as at least 200 grams, such as at least 250 grams, such as at least 300 grams, such as at least 350 grams, such as at least 400 grams, such as at least 450 grams, such as at least 500 grams, such as at least 550 grams. In certain embodiments, the composition may contain less than 550 grams of creatine, such as less than 500 grams, such as less than 450 grams, such as less than 400 grams, such as less than 350 grams, such as less than 300 grams, such as less than 250 grams, such as less than 200 grams, such as less than 150 grams, such as less than 100 grams.

In certain embodiments, the creatine included in the protein building composition may be formulated as lipid multiparticulates or in lipid multiparticulate form. For example, lipid multiparticulates (LMPs) are known. See for example, EP1030687, U.S. Pat. No. 6,572,892, EP1827382, U.S. Pat. Nos. 7,235,260, 7,887,844, EP1691787, U.S. Pat. No. 7,625,507. Administration of creatine into low pH environments, such as the stomach, can degrade the efficacy and potency of creatine. Thus, providing creatine in lipid multiparticulate form (LMP) as provided herein, offers protection of the creatine in acid rich environments, such as the stomach. Accordingly, the creatine in LMP form provided herein, may offer additional benefits and provide better efficacy as compared to other creatine-containing compositions.

Furthermore, U.S. patent publication no. 2018/0125863, incorporated by reference herein in its entirety, describes certain oral formulations containing an active ingredient incorporated with a lipid matrix. Accordingly, in certain embodiments, the protein building composition or components of the protein building composition, such as creatine, can be formulated in combination with a lipid matrix according to those disclosed in U.S. patent publication no. 2018/0125863.

Additionally, U.S. patent publication no. 2017/0354599, incorporated by reference herein in its entirety, describes certain formulations containing an active ingredient incorporated with a lipid matrix. Accordingly, in certain embodiments, the protein building composition or components of the protein building composition, such as creatine, can be formulated in combination with a lipid matrix according to those disclosed in U.S. patent publication no. 2017/0354599.

In certain embodiments, the creatine may be provided in a composition that contains a lipid matrix. The lipid matrix can include a) at least one low flow point excipient, b) at least one high flow point excipient, c) at least one low-flow point surfactant, and c) optionally an antioxidant. In one embodiment, creatine comprises at least 10 wt % of the lipid matrix composition, such as at least 20 wt %, such as at least 30 wt %, such as at least 40 wt %, such as at least 50 wt %. In one embodiment, the at least one low flow point excipient comprises at least 10 wt % of the lipid matrix composition, the at least one high flow point excipient comprises at least 5 wt % of the lipid matrix composition, and the at least one low-flow point surfactant comprises at least 10 wt % of the lipid matrix composition. In one embodiment, the lipid matrix composition containing creatine may comprise a plurality of particles that are solid or semi-solid at ambient temperature and have a generally spherical shape.

In one embodiment, the lipid matrix may contain components selected from the group consisting of fatty alcohols, fatty acids, fatty acid esters of glycerol, glycols and poly glycols, fatty acid esters of polyglycerol, polyglycolized glycerides, C8-C18 triglycerides, stearoyl polyoxylglycerides, lauroyl macrogol-32 glycerides, caprylocaproyl macrogol-8 glycerides, oleoyl macrogol-6 glycerides, linoleoyl macrogol-6 glycerides, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, propylene glycol monocaprylate esters, propylene glycol dicaprate/dicaprylate esters, propylene glycol heptanoate, propylene glycol monostearate, propylene glycol monooleate, propylene glycol monopalmitate, propylene glycol monomyristate, esterified alpha-tocopheryl polyethylene glycol succinate, propylene glycol monolaurate esters, polyoxyl 35 castor oil, polyoxyl 40 hydrogenated castor oil, lecithins, vitamin E, tocopheryl polyethylene glycol succinate (TPGS), sugar fatty acid esters, sorbitan fatty acid esters, polyoxyethylene sorbitan fatty acid esters, polyoxyethylene-polyoxypropylene copolymers, propylene glycol, triacetin, isopropyl myristate, diethylene glycol monoethyl ether, polyethylene glycol, glycerol, rosemary extract, ascorbic acid, ascorbyl palmitate, butylated hydroxyanisole (BHA), butylated hydroxytoluene (BHT), and mixtures and combinations thereof.

In some embodiments, the creatine is contained within a lipid matrix containing stearyl alcohol, stearic acid, candelilla wax, and lecithin. In certain embodiments the lipid matrix may contain from about 40% about 60% by weight of creatine, such as about 50% by weight of creatine. In certain embodiments, the lipid matrix may contain from about 15% to about 25% by weight of stearyl alcohol, such as about 17% by weight of stearyl alcohol. In some embodiments, the lipid matrix may contain from about 10% to about 20% by weight of stearic acid, such as from about 15% by weight of stearic acid. In some embodiments, the lipid matrix may contain from about 10% to about 20% of a suitable wax, such as candelilla wax, such as from about 15% by weight of a suitable wax. In some embodiments, the lipid matrix may contain from about 1% to about 3% of a lecithin, such as about 2% of a lecithin, such as soy lecithin.

In certain embodiments, the lipid matrix containing creatine may be formed and then formulated into one or more particles having a generally spherical shape and a mean diameter ranging from about ranging from 40 μm to 3000 μm, such as from about 100 μm to 2000 μm, such as from about 300 μm to 1000 μm.

The different components of the protein building composition, such as L-carnitine and creatine, can be present in the protein building composition at various ratios depending upon the particular application and the desired result. The weight ratio between the amino acid derivative, such as L-carnitine, and the nitrogenous organic acid, such as creatine, can generally be from about 10:1 to about 1:3, such as from about 9:1 to about 1:2, such as from about 8:1 to about 1:1, such as from about 7:1 to about 1:1, such as from about 6:1 to about 1:1, such as from about 5:1 to about 1:1, such as from about 4:1 to about 1:1, such as from about 3:1 to about 1:1, such as from about 2:1 to about 1:1.

The manner in which the L-carnitine and creatine are combined to produce the protein building composition can vary depending upon the particular application and the desired results. In one aspect, for instance, the L-carnitine and the creatine are both solids that are blended together. For example, the creatine can be in pure form or in a lipid multiparticulate form as described above. The L-carnitine, on the other hand, can be in pure form or can also be in lipid multiparticulate form. As used herein, a pure form indicates that the solid particles contain primarily the amino acid component and/or the nitrogenous organic acid component. For instance, each particle can contain the amino acid component and/or the nitrogenous organic acid component in an amount greater than about 80% by weight, such as in an amount greater than about 85% by weight, such as in an amount greater than about 90% by weight, such as in an amount greater than about 95% by weight. The particles containing creatine and the particles containing the L-carnitine can be combined together and blended. Once the L-carnitine and the creatine are blended, the protein building composition can be placed in any suitable vehicle for delivery to a patient or user.

In one embodiment, the protein building composition can be placed in a vehicle that is acid resistant. For instance, the protein building composition can be placed in a capsule that protects the protein building composition from the low pH environment of the stomach. The capsule, for instance, can be designed to release after an extended period of time, protecting the protein building composition from degradation in the stomach. In this manner, the protein building composition is released in the small intestines where it can be more efficiently absorbed into the bloodstream.

Using an acid resistant capsule can provide various advantages and benefits. For instance, by using an acid resistant capsule, the amino acid component and the nitrogenous organic acid component can be present in relatively pure forms within the capsule without any significant degradation occurring in the stomach. Using relatively pure forms of the different components not only reduces costs but also reduces complexity.

The acid resistant capsule can include a capsule shell made from two co-axial, telescopically-joined parts. The two parts can be referred to as a body and a cap. The side wall of each of the parts is generally greater than the capsule diameter. The capsule caps and bodies are telescopically joined together so as to make their side walls partially overlap and obtain a hard capsule shell.

In one embodiment, the capsule shell can be formed from an aqueous composition that contains an aqueous solvent, and at least one water soluble, film forming polymer. The water soluble, film forming polymer can be hydroxypropyl methylcellulose alone or in combination with other film forming polymers such as gelatin, pullulan, polyvinyl alcohol, a starch derivative such as hydroxypropyl starch, or the like.

In one aspect, the film forming polymer is combined with a gum. For instance, the gum can be gellan gum. The gum can be present in relation to the film forming polymer such as the hydroxypropyl methylcellulose at a weight ratio of from about 4 to about 15 parts of the gum per 100 parts of the hydroxypropyl methylcellulose, such as from about 4.5 to about 8 parts of the gum per 100 parts of the hydroxypropyl methylcellulose, such as from about 4.5 to about 6 parts of the gum per 100 parts of the hydroxypropyl methylcellulose.

In one embodiment, the composition used to produce the acid resistant capsule can contain a gelling aid. The gelling aid can be a potassium, sodium, or calcium salt.

Acid resistant hard capsules made according to the present disclosure can have a certain acid resistance when tested using the apparatus and procedure disclosed in the disintegration test for dosage forms of USP-30 which uses a simulated gastric fluid at 37±2° C. in a basket/rack assembly. The acid resistant capsule of the present disclosure, for instance, can display an acid resistance such that the capsule does not leak for at least one hour when placed in a simulated gastric fluid at a pH of 1.2 in accordance with test USP-30.

The protein building composition can be loaded into the acid resistant capsule. In one aspect, the protein building composition is a solid. For instance, the protein building composition can be a solid containing the amino acid component combined with the nitrogenous organic acid component. In one aspect, the amino acid component and the nitrogenous organic acid component are powders that have been blended together. The amino acid component can comprise an L-carnitine or derivative powder that is in substantially pure form and the nitrogenous organic acid component can comprise a carnitine or derivative powder that is in substantially pure form.

Capsules used in accordance with the present disclosure generally include two cooperating parts. For instance, the capsule shell can have an elongated body formed from a first part or component (capsule cap) that cooperates with and overlaps with a second part or component (capsule body). In one aspect, the capsule can be banded. A banded capsule is one that has an additional closure or seal where the two parts of the capsule come together. The band that is formed on the capsule seals the joint between the capsule cap and the capsule body. The band can be made from various materials, such as natural polymers. For instance, the band can be made from liquid gelatin, cellulose, or mixtures thereof.

The use of a banded capsule can further prevent acid breakdown of the capsule in the stomach. Banded capsules prevent the capsules from leaking, especially when containing any liquid components. Banded capsules also reduce oxidation of the contents. Once banded, the capsule is almost impossible to open and re-close without leaving evidence of tampering.

In an alternative embodiment, the protein building composition can be in the form of a suspension contained within the acid resistant capsule.

For example, the L-carnitine can be in liquid form. The creatine, on the other hand, can be in solid form and can be suspended within the liquid L-carnitine. The L-carnitine, for instance, can be dissolved in a liquid carrier, such as polar solvent. For example, in one embodiment, the L-carnitine can be contained in water. The solid creatine particles, on the other hand, can be added to the liquid L-carnitine at desired amounts for creating a suspension.

Once formed into a suspension, the protein building composition can then be placed in an acid resistant capsule.

In some embodiments, the protein building composition may optionally include an amino acid in combination with L-carnitine and creatine. The amino acid may be leucine and any derivatives, metabolites, and/or salts thereof.

In one embodiment, the composition may comprise one or more vitamins. In one particular embodiment, the composition may comprise vitamin D3. Vitamin D can regulate muscle contractility. Other vitamins may include but are not limited to vitamin A, vitamin B1, vitamin B2, vitamin B3, vitamin B6, vitamin B9, vitamin B12, vitamin C, vitamin E, vitamin K, riboflavin, niacin, folic acid, pyridoxine, thiamine, pantothenic acid, biotin, and any combinations thereof. The composition may further comprise other minerals, herbs, botanicals, and essential fatty acids. In one embodiment, the protein building composition may comprise magnesium and/or salts thereof.

In one embodiment, the one or more vitamins may be present in the composition in an amount of about 1 to about 5,000 IU per dose, such as about 10 to about 2,500 IU per dose, such as about 50 to about 1,500 IU per dose, such as about 100 IU to about 1,000 IU per dose, such as about 250 IU to about 750 IU per dose, such as about 300 IU to about 600 IU per dose.

In one embodiment, the amino acids, amino acid derivatives, and/or organic acids and derivatives and/or salts thereof may be included in the composition as free form organic compounds. Alternately, the components may be included in the composition as intact proteins and/or other macromolecules. In a further embodiment, the amino acids, amino acid derivatives, and/or organic acids and derivatives and/or salts thereof may be included in the composition as a combination of free form organic compounds and intact protein and/or other macromolecules.

The present disclosure is also directed to methods of administering the protein building composition disclosed herein. For example, administration of L-carnitine in combination with creatine has been discovered to increase protein synthesis, which may lead to increased functional strength in mammals. In some embodiments, the administration of L-carnitine in combination with creatine may provide synergistic benefits. Indeed, lesser amounts of creatine can be utilized when creatine is administered in combination with L-carnitine given the synergistic benefits discovered herein. In order to increase muscle protein synthesis and/or functional strength, the present disclosure is directed to a method of administering to a mammal an effective amount of a protein building composition. The above advantages and benefits may be realized without any adverse consequences. The protein building composition can be administered regularly, such as at least two to four times a week. For instance, the protein building composition may be administered to the mammal at least everyone to three days. Further, the protein building composition may be administered once a day or more than one time per day. For instance, the protein building composition may be administered to the mammal one to four times per day. In one particular embodiment, the protein building composition is administered daily.

The protein building composition can be administered to the mammal in any suitable form using any suitable administration route. For example, the composition can be administered orally alone, in combination with a food composition, or as part of a food composition. The composition may also be part of a dietary supplement or as a nutraceutical composition. The composition may be administered alone as a dietary supplement in the form of a monolithic enteric capsule having a particular gastrointestinal release profile.

In certain embodiments the protein building composition can be administered via a delivery system designed to improve the bioavailability of the protein building composition. For example, U.S. patent Publication No. 2016/0256399, incorporated herein by reference in its entirety, discloses certain delivery systems, including enteric drug delivery systems, that are capable of increasing the bioavailability of active ingredients, such as L-carnitine and creatine or the protein building composition of the present disclosure, via an oral monolithic enteric capsule. Indeed, the use of such enterically coated capsules may provide a modified release profile within the gastrointestinal tract of a mammal. In general, any form of the protein building composition as described above can be administered to a mammal in accordance with the present disclosure. Such forms include a solid/solid blend of L-carnitine and creatine, a suspension where solid creatine is suspended in a liquid L-carnitine, or the like. In still another embodiment, the creatine and L-carnitine can be delivered separately and can be administered separately to the mammal or mixed together in desired proportions and administered to a mammal.

Generally, capsules are monolithic dosage forms widely used in the nutraceutical field for oral administration to mammals. Advantages of capsules over other conventional dosage forms (such as tablets or liquids) may include better patient compliance, greater flexibility in dosage form and design, taste masking, and less expensive manufacturing processes.

Capsules normally consist of a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. See, e.g., U.S. Pat. Nos. 5,264,223, 5,756,123, and 5,756,123. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. See, e.g., EP 0401832, U.S. Pat. Nos. 3,493,407, 4,001,211, and 3,617,588, GB 1310697, and WO 2008/050209. The aforementioned manufacturing processes involve the use of solutions of the different ingredients that are needed for the making the telescoping fit hard capsule shells.

Hard capsules may be filled with active ingredients, such as the protein building composition described herein, via procedures known in the art. Typically, active ingredients are combined with various compatible excipients for ease of fill. The resulting fill may be a dry powder, a granulation, pellets, lipid pellets, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets.

For example, in some embodiments, provided is a nutraceutical composition that contains a monolithic enteric capsule filled with the protein building composition. In some embodiments, the protein building composition or the components of the protein building composition have not been enterically coated for modified release or gastric protection.

Certain embodiments comprise a monolithic enteric capsule made by dip molding from an aqueous composition comprising hydroxypropyl methyl cellulose acetate succinate (HPMCAS) polymer dispersed in water, wherein the polymer is present in an amount ranging from about 15% to about 25% by weight of the total weight of the aqueous composition; at least one dispersant in an amount ranging from about 0.5% to about 2% by weight of the total weight of said aqueous composition; at least one gelling agent present in an amount ranging from about 0.1% to about 5% by weight of the total weight of said aqueous composition; and water; and wherein the dispersed polymer is partially neutralized with at least one alkaline material.

Certain embodiments comprise a monolithic enteric capsule made with a non-salified functional polymer, said polymer being present in an amount ranging from about 50% to about 75% by weight of the total weight of the empty capsule; at least one processing aid present in an amount ranging from about 10.5% to about 20% by weight of the total weight of the empty capsule; and water present in an amount ranging from about 1% to about 20% by weight over the total weight of the empty capsule.

Certain embodiments comprise a monolithic enteric capsule comprising cellulose acetate phthalate (CAP), in an amount ranging from about 40% to about 70% by weight; and at least one processing aid selected from polyoxyethylene-polyoxypropylene-polyoxyethylene tri-block polymers and mixtures thereof, and having an average molecular weight ranging from about 1000 to about 20000 and a polyoxyethylene ratio ranging from about 10% to about 80%, in an amount ranging from about 15% to about 49% by weight.

In certain embodiments, the monolithic enteric capsule for use in any of the systems and/or methods of the present disclosure lacks internal excipients. In certain embodiments, the monolithic enteric capsule remains substantially intact in the stomach.

In certain embodiments, the monolithic enteric hard capsule may comprise an enteric coating for modified release or gastric protection. In some embodiments, less than about 10% of the protein building composition is released from the monolithic enteric capsule after about 2 hours in a pH of about 1.2. In some embodiments, about 80% of the protein building composition is released from the monolithic enteric capsule after about 30 min at a pH of 6.8. In some embodiments, more than about 95% of the protein building composition is released in the intestine.

In certain other embodiments, the protein building composition can be administered orally as a solid, liquid, suspension, or gas. The composition may be administered via buccal or sublingual administration. In one embodiment, the protein building composition may be administered as a capsule, tablet, caplet, pill, troche, drop, lozenge, powder, granule, syrup, tea, drink, thin film, seed, paste, herb, botanical, and the like. In addition to being administered orally, the supplement dose containing the protein building composition can also be administered using other routes including intranasal, intravenous, intramuscular, intragastric, and the like.

When the protein building composition is combined with a food or beverage composition, the food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

A food composition ingested by the mammal in addition to the protein building composition may also be rich in L-carnitine and/or creatine. The protein building composition of the present disclosure, for instance, is intended to provide additional L-carnitine and/or creatine in addition to the normal amounts contained in a standard diet and/or the amounts produced by the body.

The mammal treated in accordance with the present disclosure can comprise any suitable mammal. For instance, the mammal may be human or canine. The protein building composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood. For instance, the active mammal may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like.

The protein building composition may be administered to the mammal according to the present disclosure regardless of the frequency, intensity, or type of physical activity performed by the mammal. The mammal may participate in physical activities with various MET values. In one embodiment, the mammal may regularly participate in light to intense physical activity. Light physical activity may have a MET of from about 3 MET to about 6 MET. Moderate physical activity may have a MET of from about 6 MET to about 10 MET. Intense physical activity may have a MET of about 10 MET or greater. In another embodiment, the mammal may infrequently participate in physical activity. In yet another embodiment, the mammal may lead a sedentary lifestyle, wherein the mammal may rarely or never participate in physical activity. In a sedentary lifestyle, a mammal may participate mainly or exclusively in sedentary activities.

The protein building composition may be administered to the mammal before, during, or after a period of physical activity. Alternately, the composition may be administered to the mammal before, during, or after a period of sedentary activity. For instance, the composition may be administered to the mammal during an extended period of bed rest or other extended period of inactivity.

The protein building composition is administered in an amount sufficient to increase muscle protein synthesis, increase functional strength, or increase both muscle protein synthesis and functional strength without requiring the mammal to participate in physical activity.

Muscle protein synthesis, in one embodiment, can be determined by monitoring the biomarkers, mTOR expression and phosphorylation and its related upstream and downstream proteins in the pathway, in skeletal muscle. Specifically, mTOR expression can be determined and recorded before and after a period of activity. For a mammal treated in accordance with the present disclosure, mTOR expression before and after a period of time may vary by more than 10%, such as by more than 20%, such as by more than 40%, such as by more than 60%, such as by more than 80%, such as by more than 100%, such as by more than 150%, such as by more than 200%.

In one embodiment, the mammals treated in accordance with the present disclosure may have total mTOR values after a period of activity that are at least 10%, such as at least 20%, such as at least 30%, such as at least 40%, such as at least 50%, such as at least 60%, such as at least 100% greater than the same mammal that is not administered the protein building composition.

Protein synthesis can be monitored by androgens, androgen receptors, insulin, IGF-1, IGF-1 receptors and any known stimulator of protein synthesis.

In one embodiment, the compositions of the present disclosure may contain other amino acids, including but not limited to alanine, arginine, asparagine, aspartate, cysteine, glutamic acid, glutamine, glycine, proline, serine, tyrosine, histidine, isoleucine, lysine, methionine, phenylalanine, threonine, tryptophan, valine, and any combinations thereof.

The compositions and methods of the present disclosure can also increase the bioavailability of the amino acid component and/or the nitrogenous organic acid component when administered to a mammal in comparison to only administering one of the above components to the mammal. For example, by combining the amino acid component with creatine, the bioavailability of creatine can be increased in comparison to only administering creatine. Thus, creatine can be administered to a mammal with greater efficiency and without having to increase doses that, in some circumstances, may cause an uncomfortable burning sensation. The increased bioavailability of one or both components can further be enhanced when one or both of the components are in multiparticulate form and/or in an acid resistant capsule.

The protein building composition of the present disclosure may further comprise one or more excipients. Exemplary but non-limiting excipients include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one embodiment, the protein building composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the protein building composition in order to improve the handling properties of the composition. For instance, the stabilizer package may allow the composition to have better flow properties, especially when in granular form.

The present disclosure may be better understood with reference to the following examples.

### Example No. 1

The effects of creatine and L-carnitine on skeletal muscle protein synthesis in primary human myoblast cultures was studied.

Human skeletal muscle myoblasts (HSMM) were obtained from Lonza and were maintained at 37° C. (95% O2-5% CO2) in Lonza growth media. For experimental procedures, the myoblasts were seeded in 12-well plates and treated with CrMH only (1.0 mM, 0.1 mM, 0.05 mM, 0.01 mM, 0.005 mM, 0.001 mM, and 0.0005 mM), L-Car only (0.1 mM, 0.05 mM), and variable proportions of CrMH and L-Car (1.0/1.0 mM, 0.5/0.5 mM, 0.1/0.1 mM, 0.05/0.05 mM, 0.005/0.05 mM, 0.0005/0.05 mM, 0.0001/0.05 mM). Protein synthesis and anabolic signaling measures were made at the 24 h time point for all conditions. After 22 h of stimulus, cells were subjected to a 1.5 h serum-free starve, after which the media and appropriate stimulus were reapplied along with puromycin. After 30 minutes of puromycin treatment, cells were lysed and protein was extracted. Protein synthesis was evaluated by measuring puromycin incorporation during the experiment using the SUnSET technique. Anabolic signaling was determined as the quantity of pAKT and pRPS6 normalized to GAPDH.

Recombinant human IGF-1 and DMEM-F12, Tryp-LE, Horse Serum and PBS were purchased from Life Tech. Puromycin and α-puromycin Ab were purchased from Sigma (St. Louis, Mo.). Phospho-Akt and total Akt and Phospho-S6K1 and total S6K1 were purchased from Cell Signalling Technologies. Tissue lysates were solubilized in Laemmli buffer (BioRad) and separated by SDS-PAGE using precast Tris.HCl gels (BioRad). Protein was transferred to polyvinylidene fluoride membranes (BioRad).

The tested compositions are shown in Table 1.

TABLE 1
L-Carnitine and Placebo Supplement Compositions

| | Active Ingredients | Other Ingredients |
|---|---|---|
| Carnitine Sample | L-carnitine (500 mg) | Stearyl Alcohol |
| | | Stearic Acid |
| | | Candelilla Wax |
| | | Soy Lecithin |
| Creatine Sample | Creatine monohydrate (500 mg) | Stearyl Alcohol |
| | | Stearic Acid |
| | | Candelilla wax |
| | | Lecithin (Soy) |
| Placebo | | Stearyl Alcohol |
| | | Stearic Acid |
| | | Candelilla Wax |
| | | Lecithin (Soy) |

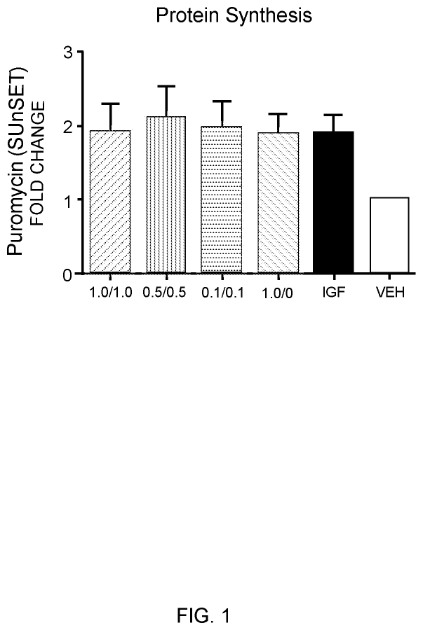

FIG. 1 depicts protein synthesis evaluated according to puromycin fold change on the vertical axis and sample combinations of certain amounts of the L-carnitine and creatine samples on the horizontal axis. As shown, the tested combinations include amounts of creatine and L-carnitine in mM amounts. For example, FIG. 1 tested 1.0/1.0 mM of creatine/L-carnitine, 0.5/0.5 mM of creatine/L-carnitine, 0.1/0.1 mM of creatine/L-carnitine, 1.0/0.0 mM of creatine/L-carnitine, IGF, and VEH.

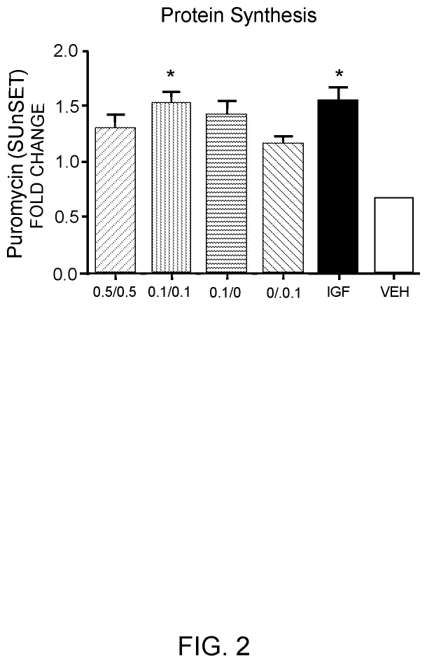

FIG. 2 depicts protein synthesis evaluated according to puromycin fold change on the vertical axis and sample combinations of certain amounts of the L-carnitine and creatine samples on the horizontal axis. As shown, the tested combinations include amounts of creatine and L-carnitine in mM amounts. For example, FIG. 2 tested 0.5/0.5 mM of creatine/L-carnitine, 0.1/0.1 mM of creatine/L-carnitine, 1.0/0.0 mM of creatine/L-carnitine, IGF, and VEH.

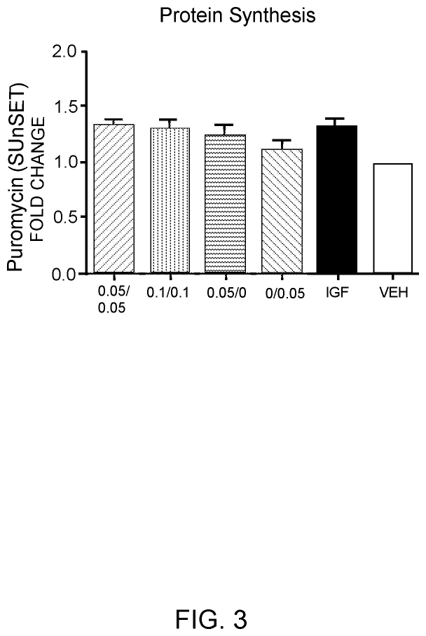

FIG. 3 depicts protein synthesis evaluated according to puromycin fold change on the vertical axis and sample combinations of certain amounts of the L-carnitine and creatine samples on the horizontal axis. As shown, the tested combinations include amounts of creatine and L-carnitine in mM amounts. For example, FIG. 3 tested 0.5/0.5 mM of creatine/L-carnitine, 0.1/0.1 mM of creatine/L-carnitine, 0.05/0 mM of creatine/L-carnitine, 0/0.05 of mM of creatine/L-carnitine, IGF, and VEH.

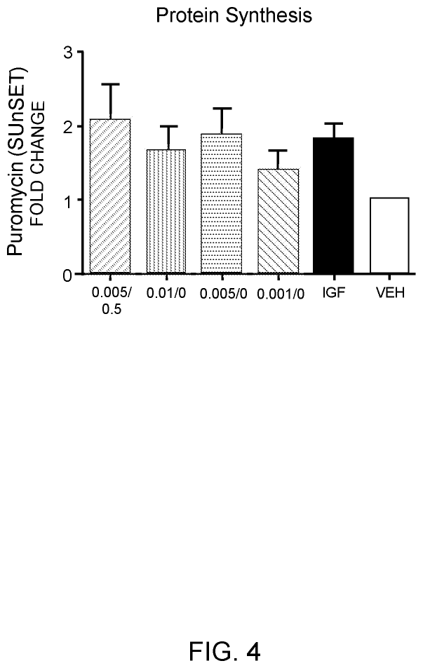

FIG. 4 depicts protein synthesis evaluated according to puromycin fold change on the vertical axis and sample combinations of certain amounts of the L-carnitine and creatine samples on the horizontal axis. As shown, the tested combinations include amounts of creatine and L-carnitine in mM amounts. For example, FIG. 4 tested 0.005/0.5 mM of creatine/L-carnitine, 0.01/0 mM of creatine/L-carnitine, 0.005/0 mM of creatine/L-carnitine, 0.001/0 of mM of creatine/L-carnitine, IGF, and VEH.

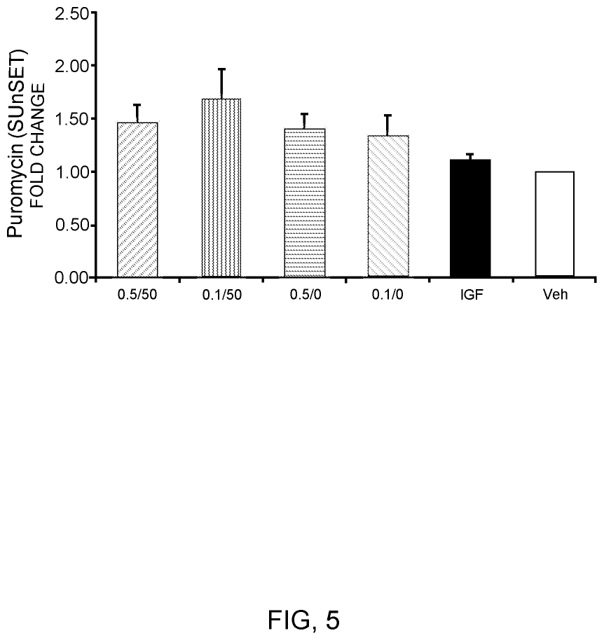

FIG. 5 depicts protein synthesis evaluated according to puromycin fold change on the vertical axis and sample combinations of certain amounts of the L-carnitine and creatine samples on the horizontal axis. As shown, the tested combinations include amounts of creatine and L-carnitine in mM amounts. For example, FIG. 5 tested 0.5/50 mM of creatine/L-carnitine, 0.1/50 mM of creatine/L-carnitine, 0.1/0 mM of creatine/L-carnitine, IGF, and VEH.

As shown in the FIGS. 1-5 provided herein the following combinations of creatine and L-carnitine showed synergistic effects on skeletal muscle protein synthesis: 0.1 mM/0.1 mM of creatine/L-carnitine, 0.05 mM/0.05 mM of creatine/L-carnitine, and 0.005 mM/0.05 mM of creatine/L-carnitine. Accordingly, certain combinations of creatine and L-carnitine may provide synergistic effects when administered according to example embodiments of the present disclosure.

Further, it was surprisingly discovered the lower amounts of creatine and L-carnitine may be utilized in order to provide synergistic effects. Utilization or administration of lower amounts of creatine and/or L-carnitine may provide numerous benefits including reduced product costs and reduction in unwanted side effects. Other benefits also include an increase in overall muscle function and performance utilizing lower amounts of creatine given the synergistic effects observed when lower amounts of creatine are administered in combination with L-carnitine.

### Example No. 2

The effect of creatine LMP release and L-carnitine release were studied.

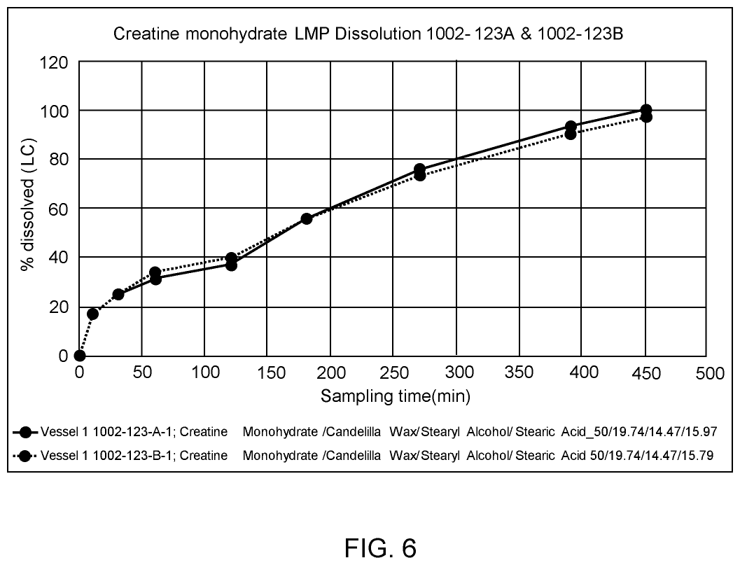

FIG. 6 depicts a dissolution profile for two samples of creatine monohydrate LMP. The creatine monohydrate LMP contains creatine monohydrate, candelilla wax, stearyl alcohol, and stearic acid. The vertical axis provides the percentage dissolved and the horizontal axis provides the sampling time in minutes. As shown, the creatine LMP is less than about 40% dissolved at 50 minutes and is about 60% dissolved after 200 minutes.

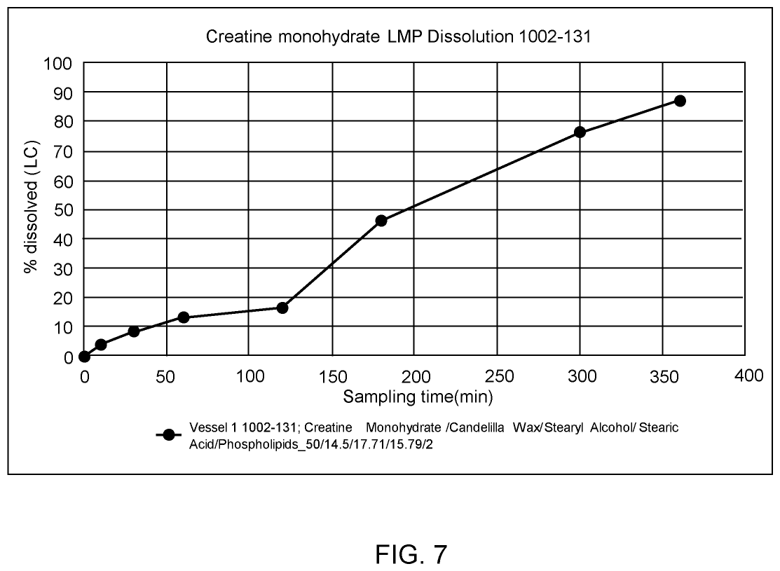

FIG. 7 depicts a dissolution profile for creatine monohydrate LMP. The creatine monohydrate LMP contains creatine monohydrate, candelilla wax, stearyl alcohol, stearic acid, and phospholipids. The vertical axis provides the percentage dissolved and the horizontal axis provides the sampling time in minutes. As shown, the creatine LMP at 50 minutes less than about 15% of the creatine LMP is dissolved. Further, about 50% of the creatine LMP is dissolved at around 200 minutes.

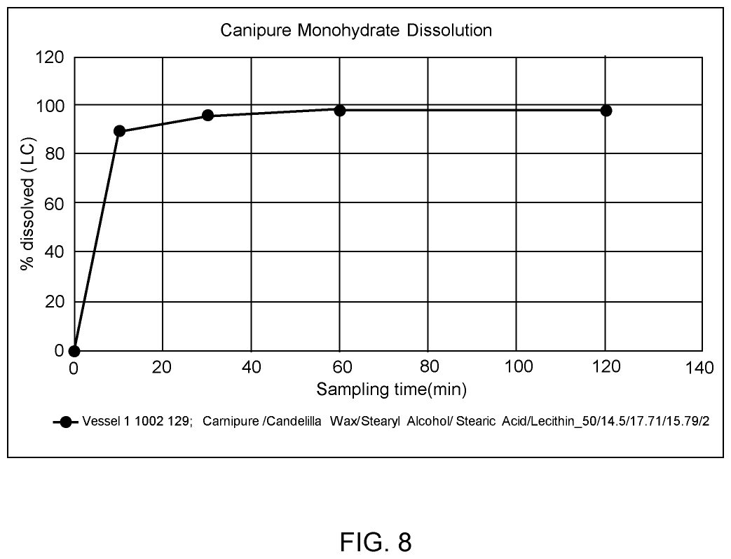

FIG. 8 depicts a dissolution profile for L-carnitine monohydrate LMP. The L-carnitine monohydrate contains L-carnitine, candelilla wax, stearyl alcohol, stearic acid, and lecithin. The vertical axis provides the percentage dissolved and the horizontal axis provides the sampling time in minutes. As shown, almost 90% of the L-carnitine LMP is dissolved at 20 minutes of testing.

As shown by the dissolution profiles, creatine monohydrate LMP provides a more steady release profile over time and a slower release profile during the first 0-100 minutes of testing. Furthermore, lower percentage amounts of creatine LMP are dissolved during the first 100 minutes of testing. Advantageously, creatine monohydrate LMP as provided in the above example, having a lower percentage of creatine LMP dissolved at 50-100 minutes, may provide for a suitable release profile in vitro, thus releasing or dissolving more in the intestines and not in the stomach upon administration.

These and other modifications and variations to the present disclosure may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

1. A method for increasing muscle protein synthesis, the method comprising administering to the mammal an effective amount of a protein building composition, the protein building composition comprising an amino acid component and a nitrogenous organic acid component, wherein the amino acid component comprises L-carnitine or a derivative thereof, wherein the nitrogenous organic acid component comprises creatine or a derivative thereof, wherein the protein building composition is contained in an acid resistant capsule shell, the capsule shell exhibiting resistance to leakage for at least one hour at a pH of 1.2 in a USP-30 simulated gastric fluid.

2. The method as defined in claim 1, wherein the capsule shell is made from a material comprising hydroxypropyl methylcellulose.

3. The method as defined in claim 2, wherein the material further comprises a gum, optionally wherein the gum comprises gellan gum.

4. (canceled)

5. The method as defined in claim 3, wherein the weight ratio of the gum to the hydroxypropyl methylcellulose in the material is from about 4.5 to about 15 parts of the gum per 100 parts of the hydroxypropyl methylcellulose.

6. The method as defined in claim 1, wherein the capsule shell includes a first component that overlaps a second component to form a joint and wherein the capsule shell further includes a band covering the joint.

7. The method as defined in claim 1, wherein the L-carnitine or derivative thereof comprises a liquid.

8. The method as defined in claim 7, wherein the protein building composition comprises a suspension, the creatine or derivative thereof comprising a solid that is suspended within the liquid or wherein the L-carnitine or derivative thereof is dissolved in a liquid carrier, optionally wherein the liquid further comprises a pH adjusting agent.

9. (canceled)

10. (canceled)

11. The method as defined in claim 1, wherein the protein building composition is administered orally.

12. The method as defined in claim 1, wherein the mammal is a human.

13. The method as defined in claim 1, wherein the protein building composition further contains one or more adjuvants chosen from anti-adherents, binders, coatings, colors, disintegrants, fillers, flavoring agents, glidants, lubricants, preservatives, sorbents, sweeteners, vehicles, vitamins, and the like.

14. The method as defined in claim 1, wherein ratio of L-carnitine or derivative thereof to creatine or derivative thereof is from about 10:1 to about 1:3.

15. The method as defined in claim 1, wherein the protein building composition is administered to the mammal in an amount sufficient to increase a bioavailability of the creatine in the mammal or wherein the protein building composition is administered to the mammal in an amount sufficient to increase protein synthesis in the mammal or wherein the protein building composition is administered to the mammal in an amount sufficient to increase mTOR expression in the muscles.

16. (canceled)

17. (canceled)

18. The method as defined in claim 1, wherein the creatine comprises creatine monohydrate.

19. The method as defined in claim 1, wherein the creatine or derivative thereof comprises a solid in the form of particles, the particles comprising greater than 95% by weight creatine or derivative thereof or wherein the L-carnitine or derivative thereof comprises a solid in the form of particles, the particles comprising greater than 95% by weight creatine or derivative thereof.

20. (canceled)

21. A composition for increasing muscle protein synthesis, comprising a protein building composition, the protein building composition comprising an amino acid component comprising L-carnitine or a derivative thereof and a nitrogenous organic acid component comprising creatine or a derivative thereof, wherein the protein building composition is contained in an acid resistant capsule shell, the capsule shell exhibiting resistance to leakage for at least one hour at a pH of 1.2 in a USP-30 simulated gastric fluid.

22. The composition as defined in claim 21, wherein the capsule shell is made from a material comprising hydroxypropyl methylcellulose.

23. The composition as defined in claim 22, wherein the material further comprises a gum.

24. The composition as defined in claim 23, wherein the gum comprises gellan gum.

25. The composition as defined in claim 23, wherein the weight ratio of the gum to the hydroxypropyl methylcellulose in the material is from about 4.5 to about 15 parts of the gum per 100 parts of the hydroxypropyl methylcellulose.

26. The composition as defined in claim 21, wherein the L-carnitine or derivative thereof comprises a liquid.

27. The composition as defined in claim 26, wherein the protein building composition comprises a suspension, the creatine or derivative thereof comprising a solid that is suspended within the liquid and/or wherein the L-carnitine or derivative thereof is dissolved in a liquid carrier.

28. (canceled)

29. The composition as defined in claim 27, wherein the liquid carrier comprises water an optionally a pH adjusting agent.

30. (canceled)

31. The composition as defined in claim 21, wherein the creatine or derivative thereof comprises a solid in the form of particles, the particles comprising greater than 95% by weight creatine or derivative thereof or wherein the L-carnitine or derivative thereof comprises a solid in the form of particles, the particles comprising great than 95% by weight creatine or derivative thereof.

32. (canceled)

33. The composition as defined in claim 21, wherein the capsule shell includes a first component that overlaps a second component to form a joint and wherein the capsule shell further includes a band covering the joint.
